(19) United States
(12) Patent Application Publication
(10) Pub. No.: US 2011/0200993 A1
(43) Pub. Date: Aug. 18, 2011

(54) NEW TREATMENT OF AUTOIMMUNE CONDITIONS

(75) Inventors: Emma Ahlqvist, Lund (SE); Rikard Holmdahl, Lund (SE)

(73) Assignee: REDOXIS AKTIEBOLAG, Goteborg (SE)

(21) Appl. No.: 12/743,480

(22) PCT Filed: Nov. 27, 2008
(86) PCT No.: PCT/SE2008/051362

§ 371 Date: Jul. 26, 2010

(30) Foreign Application Priority Data
Nov. 28, 2007 (SE) 0702635-4

(51) Int. Cl. C12Q 1/68; C12Q 1/02

(52) U.S. Cl. 435/6.11; 435/29

(57) ABSTRACT

The invention relates to methods and materials involved in diagnosing and treating autoimmune conditions. In particular, the invention relates to methods and materials involved in identifying agent suitable for the prophylaxis, prevention and/or treatment of an autoimmune condition. The invention further relates to methods and materials involved in diagnosing autoimmune diseases like arthritis, multiple sclerosis and inflammatory bowels disease accompanied by decreased cellular uptake of amino acids caused by defects in cellular amino acid transporters like Slc38A1 (Solute carrier family 38, member 1). The invention also relates to methods and materials involved in diagnosing, treating, preventing, or delaying the onset and ameliorating the symptoms of autoimmune conditions that are accompanied by defects in cellular amino acid transporters.

### FIELD OF THE INVENTION

The invention relates to methods and materials involved in diagnosing and treating autoimmune conditions. In particular, the invention relates to methods and materials involved in identifying agents suitable for the prophylaxis, prevention and/or treatment of an autoimmune condition. The invention further relates to methods and materials involved in diagnosing autoimmune diseases like arthritis, multiple sclerosis and inflammatory bowel disease accompanied by decreased cellular uptake of amino acids caused by defects in cellular amino acid transporters like Slc38A1 (Solute carrier family 38, member 1). The invention also relates to methods and materials involved in diagnosing, treating, preventing, or delaying the onset and ameliorating the symptoms of autoimmune conditions that are accompanied by defects in cellular amino acid transporters.

### BACKGROUND

Autoimmune conditions are conditions where a mammal's immune system starts reacting against its own tissues. Such conditions include, without limitation, arthritis, e.g. rheumatoid arthritis (RA), multiple sclerosis, inflammatory bowels disease, systemic lupus erythematosus, autoimmune uveitis, type I diabetes, bronchial asthma, septic arthritis induced by staphylococci or streptococci and cardiovascular disease involving vasculitis.

Rheumatoid arthritis is a chronic autoimmune disease of the joints, that affects 0.5-1% of the population with a 3:1 female predominance. In spite of extensive research efforts the aetiology of RA is still largely unknown. RA is a complex disease affected by multiple genes as well as environmental factors. Identification of susceptibility genes in human RA has so far been limited to those expectedly few genes that have large effects on the disease course and candidates that have been suggested due to a role in an RA related pathway or another autoimmune disease.

Mapping of disease genes in animal models of human diseases takes advantage of the unique possibilities to gain insight into disease mechanisms from hypothesis free mapping and combines them with the power of candidate association studies.

Collagen-induced arthritis (CIA) is the most extensively studied model of RA in mouse. After immunisation by a subcutaneous injection of collagen emulsified in adjuvant, mice develop a T and B-cell dependent inflammation of the peripheral joints that mimics many of the characteristics of human RA, including production of auto-antibodies.

### PRIOR ART

Tokuhiro et al. 2003 (Nature Genetics 35:341-8) reported an association between rheumatoid arthritis and the organic cation transporter gene SLC22A4 in a Japanese population. An association to RUNX1 was also reported indicating an epistatic effect between the two genes. Later studies have, however, not been able to confirm the findings of Tokuhiro et al. (Kuwahara et al. 2005, Arthritis Rheum 52: 2947-8; Orozco et al. 2006, J Rheumatol 33: 1235-9; Martinez et al. 2006, J Rheumatol 33: 842-6; Newman et al. 2005, Arthritis Rheum 52: 425-9; Barton et al. 2005, Arthritis Rheum 52: 752-8).

Peltekova et al. 2004 (Nature Genetics 36:471-475) reported a strong linkage between functional variants of the organic cation transporters OCTN1 (Slc22a4) and OCTN2 (Slc22a5) present in the IBD5 locus at 5q31 and susceptibility to Crohn disease. The significance of these findings have been further investigated (see e.g. Török et al. 2005, Gut 54: 1421-7; Waller et al. 2005 Gut 55: 809-14; Russell et al. 2006, Gut 55:114-23; Babusukumar et al. 2006, Am J Gastroenterol 101: 1354-61; Bene et al. 2006, Br J Nutr 98: 345-50; Torkvist et al. 2007, Scand J Gastroenterol 42: 200-6; Silverberg et al. 2007, Eur J Hum Genetics 15: 328-35). Results from these further studies still raise questions whether the variants of the Slc22a5 and S1ca22a5 are the causative gene variants in the IBD5 locus.

Vallejo-Illarramendi et al. 2006 (Neurobiol Dis 21: 154-64) reported an increased expression and activity of glutamate transporters EAAT1 (Slc1a3) and EAAT2 (Slc1a2) in MS. This was concluded to constitute a regulatory response of glial cells to toxic levels of glutamate in the CNS during inflammation and neuro degeneration.

Using global gene expression profiling US 20070059717 A1 provides a list of 3004 genes differentially expressed in systemic lupus erythematosus (SLE) patients. One of these genes is Slc38A1 which is shown to be down regulated in SLE patients and further suggested to be included in expression profiles aimed at diagnosing SLE. However, this is only one of 3004 genes suggested to be included in the SLE profile. The absolute majority of these genes are likely to be differentially expressed as a secondary effect of disease and therefore not suitable as candidate targets for development of therapy. Additionally a high proportion of the genes identified to differentially expressed are still annotated. Hence, there is no substantial likelihood provided by this work that the single differential expression of solute carriers, like Slc38A1 can be causative for development of SLE disease and therefore relevant as a drug target.

We have previously identified several CIA regulating loci in crosses between B10.Q and DBA/1 mice, including the Cia36 locus on chromosome 15. DBA/1 alleles in the Cia36 locus decrease production of anti-CII autoantibodies after immunisation and renders Cia36 congenic mice less susceptible to develop CIA compared to B10.Q mice (Ahlqvist et al. 2007, J Immunol 178:3084-90). In the studies where Cia36 was identified the locus was to complex and contained to many genes to make any conclusions about individual arthritis causative polymorphisms. In addition strong interaction from other loci and need for separation of neighbouring genes urged for smaller congenic fragment and more clear effect on disease for identification of single arthritis regulating genes.

### SUMMARY OF THE INVENTION

We have used the collagen-induced arthritis model to identify a new arthritis susceptibility gene, Slc38A1, thereby proving that changes in amino acid transport can be causative of autoimmune disease.

This invention describes the identification of a new susceptibility gene for autoimmune diseases and especially rheumatoid arthritis (RA). The present inventors have succeeded in reducing the Cia36 locus region to a 1 Mbp region containing 5 genes using congenic strains. One of these genes, Slc38A1, was demonstrated to have protein altering polymorphisms between the strains used. Slc38A1 is an amino acid transporter of aliphatic amino acids with a preference for glutamine. Expression of Slc38A1 was measured in lymphoid tissue with high relevance for autoimmune diseases, and expression could be demonstrated in spleen, lymph node, thymus, B-cells and T-cells. Splenocytes from the Cia36 congenic strains could be shown to have increased uptake of glutamine. It could therefore be concluded that Slc38A1 was in fact the arthritis regulating gene in mouse. The inventors then proceeded to do a candidate association study in human RA patients where they could confirm that the orthologous human gene, SLC38A1, is also significantly associated with arthritis in anti-citrulline antibody positive (anti-CCP+) and rheumatoid factor positive (RF+) male patients. Interestingly, the locus was male predominant in both mice and humans.

Slc38A1 is an amino acid transporter of aliphatic amino acids with a preference for glutamine, alanine, asparagine, histidine, serine and cysteine (Albers, et al. Pflugers Arch. 2001 October; 443(1):92-101, Chaudry et al. J. Neurosci. 2002 Jan. 1; 22(1):62-72, Mackenzie et al. J Biol Chem. 2003 Jun. 27; 278(26):23720-30). The protective allele of Slc38A1 was associated with increased glutamine uptake of spleen cells. It could also be shown that disease susceptibility could be increased by blocking of amino acid transport using a specific blocker of system A transporters to which Slc38A1 belongs. By showing that amino acid transporters like Slc38A1 can play such an important role in causing and regulating autoimmune diseases we identify the potential to use such amino acid transporters as a means to identify and diagnose autoimmune disorders like RA, to identify and diagnose subgroups of patients with autoimmune disease that is caused by altered amino acid transport and also to alter the function of these amino acid transporters with activating compound as a means of preventing and treating autoimmune diseases.

The invention provides methods and materials related to diagnosing and treating autoimmune conditions such as arthritis, RA, multiple sclerosis, inflammatory bowel diseases, including Crohn's disease and ulcerative colitis, systemic lupus erythematosus, autoimmune uveitis, type I diabetes, bronchial asthma, septic arthritis induced by staphylococci or streptococci, and cardiovascular disease involving vasculitis. Specifically, the invention provides methods and materials involved in diagnosing autoimmune conditions that are accompanied by an altered function in an amino acid transporter, preferably an amino acid transporter with a preference for glutamine, alanine, asparagine, histidine, serine and/or cysteine, as the Slc38A1 amino acid transporter. The invention further provides methods and materials involved in treating, preventing, ameliorating the severity and/or delaying the onset of autoimmune conditions that are accompanied by a decreased amino acid transporter function, and methods and materials involved in identifying agonists and antagonists of amino acid transporter activity.

Diagnosing patients having an autoimmune condition as having impaired amino acid transporter function can help clinicians determine appropriate treatments for those patients. For example, a clinician who diagnoses a patient as having impaired amino acid transporter function can treat that patient with medication that improves both the patient's autoimmune condition and amino acid transporter function. In some cases, a single medication can be used to improve a patient's level of amino acid transporter function activity such that the patient's autoimmune symptoms are reduced or relieved. Thus, treating a patient having impaired amino acid transporter function by modulating the function of cellular amino acid transporters can improve that patient's health and quality of life by, for example, reducing the symptoms or severity of symptoms associated with the autoimmune condition, or delaying the onset of the autoimmune condition.

In addition, identifying modulators, such as agonists and antagonists, of amino acid transporter function can help both clinicians and patients. For example, the methods and materials described herein can be used to identify agents that increase amino acid transporter function such that patients with impaired amino acid transporter function can be treated successfully.

The invention is based on the discovery that arthritis can be caused or aggravated by impaired amino acid transporter function. For example, development of severe arthritis symptoms in an arthritis animal model can be dependent on low amino acid transporter, like Slc38A1, function. The invention also is based on the discovery that the reduced level of amino acid transporter function responsible for arthritis susceptibility can be caused by sequence variations in amino acid transporters like Slc38A1. In addition, the invention is based on the discovery that mammals prone to develop arthritis can be protected by providing those mammals with normal levels of amino acid transporter function. For example, an animal model prone to develop severe arthritis symptoms can be rescued by providing that animal with a fully functional amino acid transporter function, like Slc38A1.

In general, the invention provides a method for assessing a mammal's susceptibility to develop an autoimmune condition, the method comprising: (a) determining the level of amino acid transporter activity in a sample of cells obtained from said mammal; (b) determining whether or not the level is less than a control level of amino acid transporter activity, wherein the control level is the average amount of amino acid transporter activity of control cells from a population of healthy mammals, and wherein the healthy mammals are from the same species as the mammal; and (c) identifying the mammal as being susceptible to develop an autoimmune condition when the level is less than the control level.

The level of amino acid transporter activity can be determined by measuring the cellular uptake of amino acids, such as glutamine, alanine, asparagine, histidine, serine or cysteine. The population can contain at least 10 healthy mammals. Step (b) can include determining whether or not the level is between 5 and 75 percent less than the control level, wherein step (c) includes identifying the mammal as being susceptible to develop an autoimmune condition when the level is between 5 and 75 percent less than the control level. Step (b) can include determining whether or not the level is between 25 and 55 percent less than the control level, wherein step (c) includes identifying the mammal as being susceptible to develop an autoimmune condition when the level is between 25 and 55 percent less than the control level.

The invention further provides a method for assessing a mammal's susceptibility to develop an autoimmune condition, the method comprising: (a) determining the level of a blood, cell tissue or synovial fluid component that reflects amino acid transporter activity in a sample obtained from said mammal; (b) determining whether or not the level is less than a control level, wherein the control level is the average amount of the component in control samples from a population of healthy mammals, and wherein the healthy mammals are from the same species as the mammal; and (c) identifying the mammal as being susceptible to develop an autoimmune condition when the level is less than the control level

The invention further provides a method for assessing a mammal's susceptibility to develop an autoimmune condition, the method comprising determining whether or not a mammal contains a mutant polypeptide of an amino acid transporter, wherein the presence of the mutant polypeptide indicates that the mammal is susceptible to develop an autoimmune condition.

The above methods can be used to determine the susceptibility to develop an autoimmune condition which can be selected from, but not limited to, arthritis, RA, multiple sclerosis, inflammatory bowel disease, systemic lupus erythematosus, autoimmune uveitis, type I diabetes, bronchial asthma, septic arthritis induced by staphylococci or streptococci, and cardiovascular disease involving vasculitis. Preferably the autoimmune condition is arthritis, such as RA, multiple sclerosis, or inflammatory bowel disease. Most preferably the autoimmune condition is arthritis, such as RA.

The invention further provides a method for diagnosing an autoimmune condition as being caused by impaired amino acid transporter function in a mammal having an autoimmune condition, the method comprising: (a) determining the level of amino acid transporter activity of a sample of cells obtained from said; (b) determining whether or not the level is less than a control level of amino acid transporter activity, wherein the control level is the average amount of amino acid transporter activity of control cells from a population of healthy mammals, and wherein the healthy mammals are from the same species as the mammal; and (c) identifying the mammal as having an autoimmune condition caused by impaired amino acid transporter function when the level is less than the control level.

The level of amino acid transporter activity can be determined by measuring cellular uptake of amino acids, such as glutamine, alanine, asparagine, histidine, serine or cysteine. The population can contain at least 10 healthy mammals. Step (a) can include contacting the sample of cells with an amino acid transporter activator. Step (b) can include determining whether or not the level is between 5 and 75 percent less than the control level, wherein step (c) includes identifying the mammal as being susceptible to develop an autoimmune condition when the level is between 5 and 75 percent less than the control level. Step (b) can include determining whether or not the level is between 25 and 55 percent less than the control level, wherein step (c) includes identifying the mammal as being susceptible to develop an autoimmune condition when the level is between 25 and 55 percent less than the control level.

The invention further provides a method for diagnosing an autoimmune condition as being caused by impaired amino acid transporter function in a mammal having an autoimmune condition, the method comprising: (a) determining the level of a blood, cell, tissue or synovial fluid component that reflects amino acid transporter activity in a sample obtained from said mammal; (b) determining whether or not the level is less than a control level, wherein the control level is the average amount of the component in control samples from a population of healthy mammals, and wherein the healthy mammals are from the same species as the mammal; and (c) identifying the mammal as having an autoimmune condition caused by impaired amino acid transporter function when the level is less than the control level.

The invention further provides a method for diagnosing an autoimmune condition caused by impaired amino acid transporter function in a mammal having an autoimmune condition, the method comprising determining whether or not the mammal contains a mutant polypeptide of an amino acid transporter, wherein the presence of the mutant polypeptide indicates that the mammal has an autoimmune condition caused by impaired amino acid transporter function.

The above methods can be used for diagnosing an autoimmune condition which can be selected from, but not limited to, arthritis, RA, multiple sclerosis, inflammatory bowel disease, systemic lupus erythematosus, autoimmune uveitis, type I diabetes, bronchial asthma, septic arthritis induced by staphylococci or streptococci, and cardiovascular disease involving vasculitis. Preferably the autoimmune condition is arthritis, such as RA, multiple sclerosis, or inflammatory bowel disease. Most preferably the autoimmune condition is arthritis, such as RA.

Another aspect of the invention provides a method for identifying an agent suitable for the prophylaxis, prevention and/or treatment of an autoimmune condition. The method comprising: (a) providing a biochemical or cellular assay comprising an amino acid transporter; (b) determining the level of amino acid transporter activity in the presence of a test agent; (c) determining whether or not the level is greater than a control level of amino acid transporter activity, wherein the control level is the amount of amino acid transporter activity in the absence of the test agent; and (d) identifying the agent as being potentially suitable for the prophylaxis, prevention and/or treatment of an autoimmune condition when the level is greater than the control level. The level of amino acid transporter activity can be determined by measuring cellular uptake of amino acids like glutamine, alanine, asparagine, histidine, serine and cysteine.

The autoimmune condition to be treated with an agent identified by the methods according to the present invention can be selected from, but not limited to, arthritis, RA, multiple sclerosis, inflammatory bowel disease, systemic lupus erythematosus, autoimmune uveitis, type I diabetes, bronchial asthma, septic arthritis induced by staphylococci or streptococci, and cardiovascular disease involving vasculitis. Preferably the autoimmune condition is arthritis, such as RA, multiple sclerosis or inflammatory bowel disease. Most preferably the autoimmune condition is arthritis, such as RA.

In another embodiment, the invention provides a method for identifying an agent that enhances amino acid transporter activity in a cell, the method comprising: (a) contacting the cell with an amino acid transporter activator and a test agent to form a test cell; (b) determining the level of amino acid transporter activity in the test cell; (c) determining whether or not the level is greater than a control level of amino acid transporter activity, wherein the control level is the amount of amino acid transporter activity in a control cell treated with the activator in the absence of the test agent; and (d) identifying the agent as enhancing amino acid transporter activity when the level is greater than the control level. The level of amino acid transporter activity can be determined by measuring cellular uptake of amino acids like glutamine, alanine, asparagine, histidine, serine and cysteine.

In another embodiment, the invention provides a method for treating a mammal having an autoimmune condition caused by impaired amino acid transporter function, the method including administering, to the animal, an agent that enhances amino acid transporter activity. The agent may be administered orally or parenterally.

In yet another embodiment, the invention features the use of an agent in the manufacture of a medicament for the treatment of an autoimmune condition caused by impaired amino acid transporter function, wherein the agent enhances amino acid transporter activity in a mammal.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a graph showing the association between arthritis severity and the Cia36 QTL containing the identified arthritis regulating amino acid transporter gene Slc38A1. Heterozygous congenic mice (triangles) had significantly lower mean arthritis scores than non-congenic mice (circles). P=0.03, n (BQ)=28, n (Cia36 het)=31. Error bars show SEM.

FIG. 2 is a graph showing the cellular uptake of glutamine in spleen cells from B10.Q mice and Cia36 congenic mice. P=0.007, n=10.

FIG. 3 is a graph showing the effect of blocking the system A transporters (including Slc38A1) on (A) mean arthritis score and (B) anti collagen antibody production. P=<0.05, n=20.

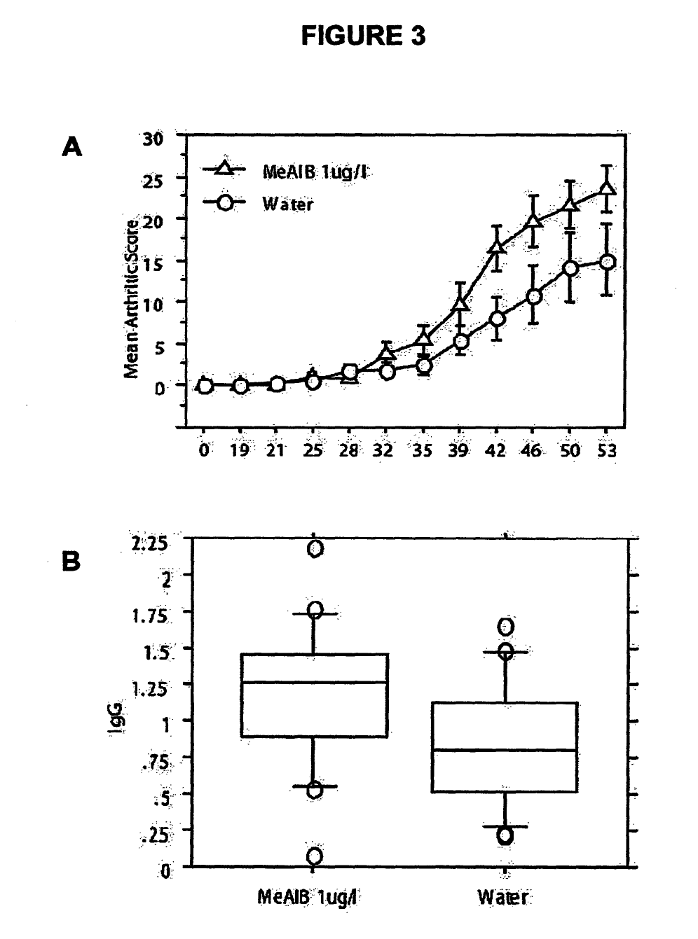

### DETAILED DESCRIPTION

The invention provides methods and materials related to diagnosing and treating autoimmune conditions. Specifically, the invention provides methods and materials involved in diagnosing mammals susceptible to develop an autoimmune condition and having impaired amino acid transporter function. In addition, the invention provides methods and materials involved in treating mammals susceptible to develop an autoimmune condition and having impaired amino acid transporter function. Further, the invention provides methods and materials involved in identifying agonists and antagonists of amino acid transporter function activity.

### 1. Diagnosing Mammals Susceptible to Develop an Autoimmune Condition

The invention provides methods for assessing a mammal's susceptibility to developing an autoimmune condition. The mammal can be a human, monkey, goat, horse, cow, pig, dog, cat, mouse, or rat. Briefly, a mammal's susceptibility to developing an autoimmune condition can be determined by examining the level of amino acid transporter activity present within the mammal's cells. This level of amino acid transporter activity can be compared with a control level, and the mammal can be classified as being susceptible to developing an autoimmune condition if the level of amino acid transporter activity in the mammal's cells is lower than the control level as further described below.

The level of amino acid transporter activity in a mammal's cells can be determined using any known method. For example, amino acid transporter activity can be assessed by measuring the cellular uptake of amino acids like glutamine, alanine, asparagine, histidine, serine and cysteine.

After determining the level of amino acid transporter activity present within the mammal's cells, this level of amino acid transporter activity can be compared with a control level of amino acid transporter activity for that particular species. The control level of amino acid transporter activity for a particular species is the average level of amino acid transporter activity measured in cells from a population of healthy members from that particular species. In the case of humans, the control level of amino acid transporter activity can be the average level of amino acid transporter activity in cells from 5, 10, 20, 30, 40, 50, or more healthy humans. If the level of amino acid transporter activity in a mammal's cells is lower than the control level, then the mammal can be classified as being susceptible to an autoimmune condition. For example, a mammal having an amino acid transporter activity that is no more than about 85, such as no more than 75, 65, 55, 45, 35, 25, 15, 5, or less, percent of the control level can be classified as being susceptible to an autoimmune condition.

Any type of sample can be used to determine the level of amino acid transporter activity in a mammal's cells. For example, the sample can be blood, cell synovial fluid, or lymph fluid containing cells, such as peripheral blood monocytes (PBMCs), having relevant amino acid transporters. Cells having amino acid transporter activity include, without limitation, whole blood lymphocytes, spleenocytes, macrophages, neutrophils, granulocytes, polymorphonuclear leukocytes, and mononuclear cells. Standard methods can be used to obtain such samples from the mammal. For example, a blood sample can be obtained by venous puncture. The sample can be subjected to any necessary standard preparatory procedures before assessing amino acid transporter activity. For example, a blood sample containing cells can be subjected to centrifugation and/or washing steps to isolate cells from which the level of amino acid transporter activity can be measured.

In another embodiment, a mammal's susceptibility to developing an autoimmune condition can be determined by examining at least a portion of the amino acid sequence of a polypeptide within one of the mammal's amino acid transporters. Any method can be used to determine the amino acid sequence of a polypeptide. For example, standard amino acid sequencing techniques can be used to determine the amino acid sequence of a purified amino acid transporter polypeptide preparation. Alternatively, the nucleic acid encoding the polypeptide can be sequenced using standard nucleic acid sequencing techniques. Once the nucleic acid sequence is determined, the amino acid sequence of the encoded polypeptide can be deduced.

If the mammal being tested contains a mutant polypeptide when compared to a comparable reference polypeptide, then that mammal can be classified as being susceptible to developing an autoimmune condition. The mutant polypeptide can be a polypeptide that contains amino acid additions, subtractions, substitutions, or combinations thereof when compared to the sequence of a comparable reference polypeptide. For example, a mutant polypeptide can be a polypeptide having any number of amino acid differences, e.g., 1, 2, 3, 4, 5, 6, 7, 8, 9, 10, 15, 20, 25, 30, 35, 40, 45, 50, 60, 70, 80, 90, 100, 125, 150, 175, 200, or more amino acid additions, subtractions, or substitutions, when compared to a comparable reference polypeptide.

Thus, in one embodiment, a human can be classified as being susceptible to developing an autoimmune condition if that human contains an amino acid transporter polypeptide having an amino acid sequence with one or more, e.g., 2, 3, 4, 5, 6, 7, 8, 9, 10, 11, 12, 13, 14, 15, or more amino acid substitutions, when compared to the amino acid sequence of control individuals.

In addition to using the presence of a mutant polypeptide to determine whether or not a particular mammal is susceptible to developing an autoimmune condition, the regulatory sequences, e.g., promoters, enhancers, and silencers, that control the expression of a polypeptide that is a part an amino acid transporter can be examined. For example, the promoter sequences that control amino acid transporter polypeptide expression can be compared to those promoter sequences that drive normal amino acid transporter polypeptide expression in healthy humans. In this case, a human having a mutated regulatory promoter sequence can be classified as being susceptible to developing an autoimmune condition.

### 2. Diagnosing Mammals Having an Autoimmune Condition Caused by Impaired Amino Acid Transporter Function

The invention provides methods for determining whether a mammal has a particular type of autoimmune condition. Specifically, a mammal can be diagnosed as having an autoimmune condition caused by impaired amino acid transporter function if that mammal has (1) clinical symptoms of an autoimmune condition and (2) cells having a level of amino acid transporter activity that is either lower than a control value or impaired function. In addition, a mammal can be diagnosed as having an autoimmune condition caused by impaired amino acid transporter function if that mammal has (1) clinical symptoms of an autoimmune condition and (2) a polypeptide that functions in the amino acid transporter that contains a mutation when compared to a comparable reference polypeptide as described herein.

For example in the case of arthritis, clinical symptoms of arthritis include, without limitation, inflammation of tendons, ligaments, joints, or bones. Symptoms of arthritis also include pain, swelling, and stiffness in the limbs that can lead to weakness, loss of mobility, and deformity in the mammal. Examples of arthritis include, without limitation, bacterial arthritis, osteoarthritis, rheumatoid arthritis (RA), collagen-induced arthritis (CIA), hexadecane-induced arthritis (HIA), pristane-induced arthritis (PIA), avridine induced arthritis, adjuvant induced arthritis, squalene induced arthritis and oil-induced arthritis (OIA).

The level of amino acid transporter activity within a mammal's cells can be assessed as described herein. Likewise, the methods and materials described herein can be used to determine whether or not a mammal contains a mutant polypeptide that is a part of an amino acid transporter.

### 3. Treating an Autoimmune Condition

The invention provides methods and materials for treating autoimmune conditions, like arthritis, e.g., an autoimmune condition caused by impaired amino acid transporter function in a mammal. Methods for treating an autoimmune condition caused by impaired amino acid transporter function include administering an agent that increases the level of amino acid transporter activity in the mammal.

Agents that increase amino acid transporter activity can be administered in any standard form using any standard method. For example, agents that increase amino acid transporter activity can be in the form of tablets or capsules, e.g., time-release capsules, that are taken orally. Alternatively, the agents can be in a liquid form and can be taken orally or by injection. The agents also can be in the form of suppositories. Further, agents that increase amino acid transporter activity can be in the form of creams, gels, and foams that can be applied to the skin. In addition, the agents can be in the form of an inhalant that is applied nasally. The agent may be administered orally, intranasally, topically or parenterally.

Agents that increase amino acid transporter activity can be administered at any dose that is sufficient to increase amino acid transporter activity in cells that have low activity. Such doses can be taken over a period of years to prevent and/or delay the progression of arthritis or to reverse the progression of arthritis. Doses can be selected based on the effectiveness and toxicity of the particular agent using standard pharmacology techniques.

4. Identifying Agents that Modify Amino Acid Transporter Activity

The invention provides methods and materials for identifying agents that modulate amino acid transporter activity. Agents that modulate amino acid transporter activity can increase or decrease amino acid transporter activity.

To identify agents that increase or decrease amino acid transporter activity, a test agent can be mixed with a sample containing cells having amino acid transporter activity. Such cells can be from humans, e.g., healthy humans or patients having an autoimmune condition, or animals, e.g., healthy animals or animals susceptible to an autoimmune condition such as those susceptible to arthritis induction. An animal is susceptible to arthritis induction if that animal develops an arthritis condition in response to treatment with an inducing agent, e.g., collagen or pristane. Such animals include those susceptible to collagen-induced arthritis (CIA), pristane—induced arthritis (PIA), hexadecane—induced arthritis (HIA), and oil—induced arthritis (OIA). The non-human animal can be any type of animal including, without limitation, monkeys, e.g., chimpanzees, horses, goats, cows, pigs, and rodents, e.g., mice and rats.

After being treated with the test agent, the level of amino acid transporter activity can be determined. The sample can be any type of sample containing a cell having amino acid transporter activity. The sample can be blood, cell lymph, or synovial fluid. The cell can be a spleenocyte, whole blood lymphocyte, purified lymphocyte, granulocyte, neutrophil, or macrophage. The sample can also be a produced biochemical system for indication of amino acid transportation.

The amino acid transporter activity determined in the presence of a test agent can be compared with the amino acid transporter activity determined in the absence of the test agent. Agents that increase amino acid transporter activity are those that, when present in the amino acid transporter assay mixture, lead to an increase in amino acid transporter activity by any amount, such as a 5, 10, 20, 30, 40, 50, 75, 100, 125, 150, 200, or more percent increase, when compared with the level of amino acid transporter activity observed in the absence of the test agent. Agents that decrease amino acid transporter activity are those that, when present in the amino acid transporter assay mixture, lead to a decrease in amino acid transporter activity by any amount, such as a 5, 10, 20, 30, 40, 50, 75, or more percent decrease, when compared with the level of amino acid transporter activity observed in the absence of the test agent.

In another embodiment, agents that increase or decrease amino acid transporter activity can be identified in an assay mixture that includes amino acid transporter, an activator, and a test agent. In these embodiments, amino acid transporter activity, determined in the presence of an activator and a test agent, can be compared with the amino acid transporter activity determined in the presence of the activator without the test agent. As described herein, agents that increase amino acid transporter activity are those that, when present in the amino acid transporter assay mixture, lead to an increase in amino acid transporter activity by any amount, such as a 5, 10, 20, 30, 40, 50, 75, 100, 125, 150, 200, or more percent increase, when compared with the level of amino acid transporter activity observed in the absence of the test agent, while agents that decrease amino acid transporter activity are those that, when present in the amino acid transporter assay mixture, lead to a decrease in amino acid transporter activity by any amount, such as a 5, 10, 20, 30, 40, 50, 75, or more percent decrease, when compared with the level of amino acid transporter activity observed in the absence of the test agent.

### 5. Non-Human Mammals Having an Impaired Amino Acid Transporter Function

Another aspect of the invention features methods and materials for providing a non-human mammal having an impaired amino acid transporter function, where the non-human mammal exhibits symptoms of an autoimmune condition. The mammal may be a monkey, goat, horse, cow, pig, dog, cat, mouse, or rat. The autoimmune condition may be arthritis, multiple sclerosis, lupus, autoimmune uveitis, type I diabetes, bronchial asthma, septic arthritis induced with staphylococci or streptococci, or cardiovascular disease involving vasculitis. For example, the non-human mammal may exhibit symptoms of arthritis. The arthritis may be induced by standard techniques known in the art and may be, e.g., adjuvant induced arthritis, collagen induced arthritis, pristane induced arthritis, hexadecane induced arthritis, avridine induced arthritis, squalene induced arthritis or oil induced arthritis.

The impaired amino acid transporter function in the non-human mammal may be indicated by a reduced amino acid transporter activity

6. Screening of Agents that Delay, Treat, or Prevent Autoimmune Conditions

In another embodiment, the invention provides a method of screening an agent to determine if the agent delays the onset of an autoimmune condition or ameliorates the symptoms of an autoimmune condition. The method includes: (a) (a) providing a nonhuman mammal having an impaired amino acid transporter function; (b) administering to the nonhuman mammal the agent; (c) inducing the autoimmune condition in the nonhuman mammal; and (d) determining if the agent have ameliorating or preventing properties on the autoimmune condition in the nonhuman mammal.

The agents to be screened can preferably be identified as having amino acid transporter modulating activity in cellular assays. Identification of agents in cellular assays may be performed by measurement of cellular uptake of amino acids, including glutamine, alanine, asparagine, histidine, serine and cysteine, in cultured cells or cells from blood or tissue samples from a mammal.

The mammal having an impaired amino acid transporter function may be obtained as described previously.

As an example, determining if the agent have ameliorating or preventing properties on arthritis may include steps such as: (a) determining a day of onset or severity of arthritis value for the nonhuman mammal; and (b) comparing the day of onset or severity of arthritis value for said nonhuman mammal with a control arthritis value. Onset and severity of arthritis can be monitored using a macroscopic scoring system, wherein 1 point is given for each swollen or red toe, 1 point for each swollen midfoot, digit, or knuckle, and 5 points for a swollen ankle, yielding a maximum score of 15 per limb and 60 total. The score can be a mean score, additive score, or maximum score. The mammal may be monitored 1 to 4 times a week for 1 to 2 months after induction of arthritis. The control day of onset and severity of arthritis value may be determined by determining severity and a day of onset of arthritis value for a control nonhuman mammal to which the agent has not been administered. The arthritis may be induced my conventional means and can be adjuvant induced arthritis, collagen induced arthritis, pristane induced arthritis, hexadecane induced arthritis, avridine induced arthritis, squalene induced arthritis, and oil induced arthritis.

The invention also provides methods to screen an agent to determine if the agent treats an autoimmune condition. The method includes (a) providing a nonhuman mammal having an impaired amino acid transporter function, where the nonhuman mammal exhibits symptoms of an autoimmune condition; (b) administering to the nonhuman mammal the agent; and (c) determining if the agent treats the autoimmune condition in the nonhuman mammal.

By way of example, in the case of arthritis, such a determining step may involve (a) calculating an arthritis score in the nonhuman mammal; and (b) comparing the arthritis score with a control arthritis score. The mammal having an impaired amino acid transporter function exhibiting symptoms of arthritis may be obtained as described previously. The control arthritis score may be determined by calculating an arthritis score for a control nonhuman mammal to which the agent has not been administered. The arthritis score may be determined using the macroscopic scoring system described previously, and may be a mean score, additive score, or maximum score. The agent may be determined to treat arthritis if the arthritis score in the nonhuman animal is less than the control arthritis score.

In another embodiment, a method of screening an agent to determine if the agent prevents an autoimmune condition is provided. The method includes: (a) providing a nonhuman mammal having an impaired amino acid transporter function; (b) administering to the nonhuman mammal the agent; (c) administering a compound known to induce the autoimmune condition to the non-human mammal; and (d) determining if the agent prevents the autoimmune condition induced by the compound in the nonhuman mammal.

As an example, in the case of arthritis, determining if the agent prevents arthritis may include evaluating said nonhuman mammal for symptoms of arthritis. Such an evaluation may occur for a period of time, e.g., for up to 20 days, up to 30 days, up to 50 days, or up to 70 days. Determining if the agent prevents arthritis may include comparing any symptoms of arthritis and their day of onset with the symptoms and day of onset of a control nonhuman mammal to which said agent has not been administered. The macroscopic scoring system as described above may be used in the evaluation and comparison. The compound known to induce arthritis may be an adjuvant, collagen, pristane, hexadecane, avridine, squalene or oil. Collagen may be type II collagen; the oil may be incomplete Freund's adjuvant; and the adjuvant may be mycobaterial-derived.

### DEFINITIONS

The term “amino acid transporter” is meant to include but not to be limited to members of the solute carrier families with potential to carry amino acids. Names of solute carriers are given in accordance with nomenclature system proposed by the Human Genome Organization.

Examples of amino acid transporters which can be used in the methods according to the invention are the following members of the solute carrier families:

(A) high affinity glutamate and neutral amino acid transporters including SLC1A1, SLC1A2, SLC1A3, SLC1A4, SLC1A5, SLC1A6, SLC1A7;

(B) facilitative GLUT transporter including, SLC2A1, SLC2A2, SLC2A3, SLC2A4, SLC2A5, SLC2A6, SLC2A7, SLC2A8, SLC2A9, SLC2A10, SLC2A11, SLC2A12, SLC2A13, SLC2A14;

(C) heavy subunits of heteromeric amino acid transporters including SLC3A1, SLC3A2;

(D) bicarbonate transporters including SLC4A1, SLC4A1, SLC4A2, SLC4A3, SLC4A4, SLC4A5, SLC4A6, SLC4A7, SLC4A8, SLC4A9, SLC4A10, SLC4A11;

(E) sodium glucose cotransporters SLC5A1, SLC5A2, SLC5A3, SLC5A4, SLC5A5, SLC5A6, SLC5A7, SLC5A8, SLC5A9, SLC5A10, SLC5A11, SLC5A12;

(F) sodium- and chloride-dependent neurotransmitter transporters including

SLC6A1, SLC6A2, SLC6A3, SLC6A4, SLC6A5, SLC6A6, SLC6A7, SLC6A8, SLC6A9, SLC6A10, SLC6A11, SLC6A12, SLC6A13, SLC6A14, SLC6A15, SLC6A16, SLC6A17, SLC6A18, SLC6A19, SLC6A20;

(G) cationic amino acid transporter/glycoprotein-associated including SLC7A1, SLC7A2, SLC7A3, SLC7A4, SLC7A5, SLC7A6, SLC7A7, SLC7A8, SLC7A9, SLC7A10, SLC7A11, SLC7A13, SLC7A14;

(H) Na+/Ca2+ exchangers including SLC8A1, SLC8A2, SLC8A3;

(I) Na+/H+exchangers including SLC9A1, SLC9A2, SLC9A3, SLC9A4, SLC9A5, SLC9A6, SLC9A7, SLC9A8, SLC9A9, SLC9A10, SLC9A11;

(J) organic cation/anion/zwitterion transporters including SLC22A1, SLC22A2, SLC22A3, SLC22A4, SLC22A5, SLC22A6, SLC22A7, SLC22A8, SLC22A9, SLC22A10, SLC22A11, SLC22A12, SLC22A13, SLC22A14, SLC22A15, SLC22A16, SLC22A17, SLC22A18, SLC22A19, SLC22A20;

(K) Na+/(Ca2+-K+) exchangers including SLC24A1, SLC24A2, SLC24A3, SLC24A4, SLC24A5, SLC24A6;

(L) multifunctional anion exchangers including SLC26A1, SLC26A2, SLC26A3, SLC26A4, SLC26A5, SLC26A6, SLC26A7, SLC26A8, SLC26A9, SLC26A10, SLC26A11;

(M) H+/amino acid symporters including SLC36A1, SLC36A2, SLC36A3, SLC36A4;

(N) System A & N, sodium-coupled neutral amino acid transporters including SLC38A1, SLC38A2, SLC38A3, SLC38A4, SLC38A5, SLC38A6; and

(0) Na+-independent, system-L like amino acid transporters including SLC43A1, SLC43A2, SLC43A3;

Preferred amino acid transporters which can be used in the methods according to the invention are:

(A) high affinity glutamate and neutral amino acid transporters including SLC1A1, SLC1A2, SLC1A3, SLC1A4, SLC1A5, SLC1A6, SLC1A7;

(B) facilitative GLUT transporter including, SLC2A1, SLC2A2, SLC2A3, SLC2A4, SLC2A5, SLC2A6, SLC2A7, SLC2A8, SLC2A9, SLC2A10, SLC2A11, SLC2A12, SLC2A13, SLC2A14;

(G) cationic amino acid transporter/glycoprotein-associated including SLC7A1, SLC7A2, SLC7A3, SLC7A4, SLC7A5, SLC7A6, SLC7A7, SLC7A8, SLC7A9, SLC7A10, SLC7A11, SLC7A13, SLC7A14;

(M) H+/amino acid symporters including SLC36A1, SLC36A2, SLC36A3, SLC36A4;

(N) System A & N, sodium-coupled neutral amino acid transporters including SLC38A1, SLC38A2, SLC38A3, SLC38A4, SLC38A5, SLC38A6; and

(O) Na+-independent, system-L like amino acid transporters including SLC43A1, SLC43A2, SLC43A3.

Even more preferred amino acid transporters which can be used in the methods according to the invention are the System A & N, sodium-coupled neutral amino acid transporters including SLC38A1, SLC38A2, SLC38A3, SLC38A4, SLC38A5, SLC38A6

Even more preferred amino acid transporters which can be used in the methods according to the invention are the system A sodium-coupled neutral amino acid transporters including SLC38A1, SLC38A2, SLC38A4.

Most preferably the amino acid transporter which can be used in the methods according to the invention is the SLC38A1 transporter.

By the term “SLC polypeptide” is meant a polypeptide encoded by the corresponding SLC gene, e.g. by the term “SLC38A1 polypeptide” is meant a polypeptide encoded by SLC38A1 gene. The SLC polypeptides are also known in the art by other names recognized by the skilled person, e.g. the SLC38A1 polypeptide is also known in the art as ATA1, GInT, NAT2, SA2, or SAT1.

The “agent” to be tested in the methods according to the invention may be a polypeptide of at least 2 amino acids, e.g., 2 to 6 amino acids, 7 to 12 amino acids, 2 to 20 amino acids, or greater than 20 amino acids, such as 50 or more amino acids. For drug screening purposes, preferred agents are chemical compounds of low molecular weight. For example, the compounds can have a molecular weight of less than about 1000 Daltons, such as less than 800, 600 or 400 Daltons in weight. If desired, the chemical compound may be a member of a chemical library. The library may comprise any number of individual members, for example, tens to hundreds to thousands to millions etc., of suitable compounds. Representative compounds include, but are not limited to, peptides, peptoids and other oligomeric compounds (cyclic or linear), and template-based smaller molecules. For example, the compounds can be benzodiazepines, hydantoins, biaryls, carbocyclic and polycyclic compounds (e.g., naphthalenes, phenothiazines, acridines, steroids etc.), carbohydrate and amino acids derivatives, dihydropyridines, benzhydryls and heterocycles (e.g., triazines, indoles, thiazolidines etc.). Preferred chemical libraries include chemical compounds of low molecular weight and potential therapeutic agents.

The invention will be further described in the following examples, which do not limit the scope of the invention described in the claims.

### EXAMPLES

### Example 1

### Reconfirmation of the Effect of the Cia36 Locus on Disease

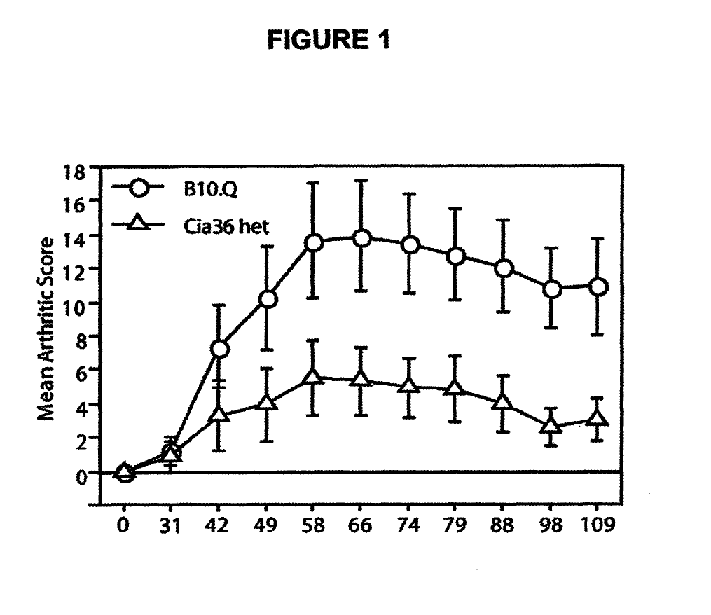

The difference in arthritis development between the arthritis susceptible BQ mice and the mice containing the protective Cia36 locus, including the amino acid transporter gene Slc38A1 is shown in FIG. 1. Heterozygous congenic mice (triangles) had significantly lower mean arthritis scores than non-congenic mice (circles).

### Example 2

### Glutamine Uptake

Cia36 mice have increased glutamine transport capacity

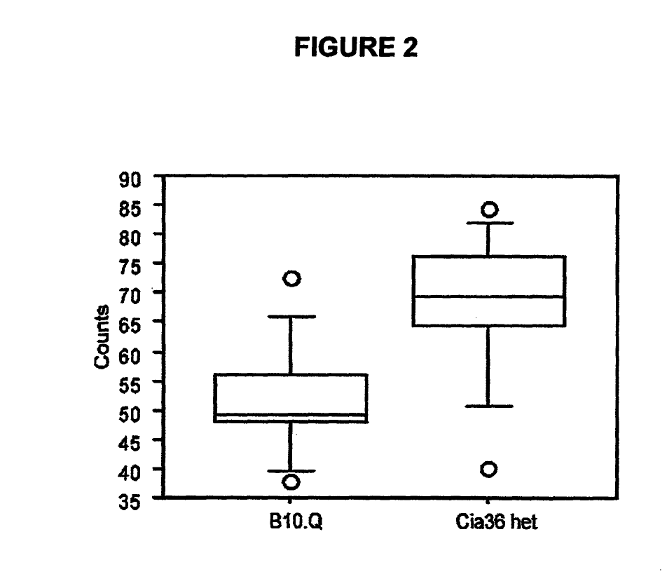

Slc38A1, previously denoted NAT2, and Slc38A2, previously denoted ATA2, are transporters of neutral amino acids with a preference for glutamine, cysteine and alanine, asparagine, histidine, serine (Gu et al. 2001, J Biol Chem 276: 24137-44) To validate if Cia36 congenic mice had an altered function of the amino acid transport system we tested the ability of cells to take up glutamine and found that a single copy of the Cia36 fragment significantly increased glutamine uptake by spleen cells in vitro. Results are presented in FIG. 2. Uptake of glutamine was significantly increased in spleen cells from Cia36 congenic mice.

### Example 3

### Effect of Blocking Transport

Blocking of transport using MeAIB, specific blocker of system A transport, increases arthritis susceptibility in mice. This confirms that disease susceptibility can be changed both by increased and decreased transporter activity.

### Example 4

### Polymorphisms in the Slc38A1 gene of the Cia36 region

In order to identify polymorphisms between the two strains that could explain the phenotypic differences we sequenced the transcribed regions of all five candidate genes in B10.Q and DBA/1. Slc38A1 was highly polymorphic with several polymorphisms including two non-synonymous coding SNPs and a splice site mutation (Table 1).

TABLE 1
Polymorphisms in the Cia 36 region

| | Transcript | Genomic | | Effect of |
|---|---|---|---|---|
| Gene | position | position | SNP alias | polymorphism |
| Arid2 | | 96126727 | rs32079390 | Intronic |
| Sfrs2ip | 4927 | 96242421 | rs31807754 | 3′UTR |
| Slc38a1 | 903/1203 | 96415143 | NT_039621.6_57702619 | M/V Non |
| | | | | synonymous coding* |
| | 842/1142 | 96416981 | NT_039621.6_57704457 | Synonymous coding |
| | 800/1100 | 96417023 | NT_039621.6_57704499 | Synonymous coding |
| | 695/995 | 96418455 | NT_039621.6_57705931 | Splice |
| | | | | site/Synonymous |
| | | | | coding |
| | 545/845 | 96420539 | Slc38a1-845 | Synonymous coding |
| | 329/629 | 96451998 | NT_039621.6_57739474 | Q/H Non |
| | | | | synonymous coding |
| | 4/304 | 96470828 | Slc38a1-304 | 5′ UTR |
| Slc38a2 | 3433 | 96516622 | Slc38a2-3433 | 3′UTR |
| | | 96527278 | rs31845248 | Intronic |
| Slc38a4 | | 96841237 | rs32219000 | Intronic |
| | | 96869584 | rs31826336 | Intronic |

### Example 5

### SNPs in SLC38A1 are Associated with Human RA

In order to investigate if the orthologous gene SLC38A1 is important in human RA, we investigated the genetic association of 20 SNPs located in the SLC38A1 region in a Swedish arthritis cohort consisting of 1500 cases and controls. We stratified the patients into 4 groups depending on sex and autoantibody status, i.e. if the patients were positive for either RFs or anti-CCP antibodies. By analysing allele frequencies we found that 9 SNPs in the SLC38A1 gene are associated with RA. The strongest associations are detected in the autoantibody positive group and the SNPs are clustered to a 24 kb region stretching from intron 11 across the UTR3′ region. Two SNPs remain significant after correcting for the number of SNPs tested; rs2289768, P=0.002, OR: 0.58, 95% CI: 0.44-0.76 and rs12368970; P=0.02, OR: 0.60, 95% CI: 0.45-0.81. Both of these SNPs have their strongest association in autoantibody positive males. Interestingly, a genomic analysis of the region using UCSC genome browser, revealed that the rs2289768 SNP is located close to exon 12 in a region which is highly conserved and has regulatory potential (Table 2).

TABLE 2
SNPs in the human SLC38A1 gene associated with RA

| | | MAF | | | | |
|---|---|---|---|---|---|---|
| | | case/ | | | | |
| SNP id | Alleles¹ | con | OR | 95% CI | P² | Subgroup |
| rs7958854 | G/A | 0.22/0.15 | 1.60 | 1.04-2.46 | 0.40 | Ab− |
| | | | | | | males |
| rs1049358 | A/G | 0.23/0.19 | 1.24 | 1.00-1.53 | 0.51 | Ab+ |
| | | | | | | females |
| rs3742058 | A/C | 0.28/0.36 | 0.68 | 0.51-0.91 | 0.15 | Ab+ |
| | | | | | | males |
| rs2053648 | A/G | 0.05/0.03 | 1.69 | 1.09-2.61 | 0.25 | Ab+ |
| | | | | | | females |
| rs3497 | A/G | 0.22/0.16 | 1.47 | 1.04-2.10 | 0.37 | Ab+ |
| | | | | | | males |
| rs3498 | G/A | 0.06/0.04 | 1.57 | 1.05-2.34 | 0.35 | Ab+ |
| | | | | | | females |
| rs12368970 | G/A | 0.24/0.35 | 0.60 | 0.45-0.81 | 0.02 | Ab+ |
| | | | | | | males |
| rs6582621 | A/G | 0.13/0.10 | 1.39 | 1.06-1.82 | 0.23 | Ab+ |
| | | | | | | females |
| rs2289768 | A/G | 0.36/0.49 | 0.58 | 0.44-0.76 | 0.002 | Ab+ |
| | | | | | | males |

MAF = minor allele frequency.
¹Minor allele first.
²corrected p-value

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for identifying an agent suitable for the prophylaxis, prevention and/or treatment of arthritis comprising:
(a) providing a biochemical or cellular assay comprising an amino acid transporter;
(b) determining the level of amino acid transporter activity in the presence of a test agent;
(c) determining whether or not the level is greater than a control level of amino acid transporter activity, wherein the control level is the amount of amino acid transporter activity in the absence of the test agent; and
(d) identifying the agent as being potentially suitable for the prophylaxis, prevention and/or treatment of arthritis when the level is greater than the control level.

2. The method of claim 1, wherein said amino acid transporter is selected from SLC1A1, SLC1A2, SLC1A3, SLC1A4, SLC1A5, SLC1A6, SLC1A7; SLC2A1, SLC2A2, SLC2A3, SLC2A4, SLC2A5, SLC2A6, SLC2A7, SLC2A8, SLC2A9, SLC2A10, SLC2A11, SLC2A12, SLC2A13, SLC2A14, SLC7A1, SLC7A2, SLC7A3, SLC7A4, SLC7A5, SLC7A6, SLC7A7, SLC7A8, SLC7A9, SLC7A10, SLC7A11, SLC7A13, SLC7A14, SLC36A1, SLC36A2, SLC36A3, SLC36A4; SLC38A1, SLC38A2, SLC38A3, SLC38A4, SLC38A5, SLC38A6; SLC43A1, SLC43A2, or SLC43A3.

3. The method of claim 2, wherein said amino acid transporter is SLC38A1.

4. The method of claim 1, wherein said level of amino acid transporter activity is determined by measuring intracellular uptake of amino acids.

5. A method for assessing a mammal's susceptibility to develop arthritis, said method comprising:
(a) determining the level of amino acid transporter activity of a sample of cells obtained from said mammal;
(b) determining whether or not said level is less than a control level of amino acid transporter activity, wherein said control level is the average amount of amino acid transporter activity of control cells from a population of healthy mammals, and wherein said healthy mammals are from the same species as said mamma; and
(c) identifying said mammal as being susceptible to develop arthritis when said level is less than said control level.

6. The method of claim 5, wherein said amino acid transporter is SLC38A1.

7. The method of claim 6, wherein said level of amino acid transporter activity is determined by measuring intracellular uptake of amino acids.

8. A method for assessing a mammal's susceptibility to develop arthritis, said method comprising:
determining whether or not a mammal comprises a mutant polypeptide that is part of an amino acid transporter, wherein the presence of said mutant polypeptide indicates that said mammal is susceptible to develop arthritis.

9. The method of claim 8, wherein said mutant polypeptide is selected from a SLC1A1, SLC1A2, SLC1A3, SLC1A4, SLC1A5, SLC1A6, SLC1A7; SLC2A1, SLC2A2, SLC2A3, SLC2A4, SLC2A5, SLC2A6, SLC2A7, SLC2A8, SLC2A9, SLC2A10, SLC2A11, SLC2A12, SLC2A13, SLC2A14, SLC7A1, SLC7A2, SLC7A3, SLC7A4, SLC7A5, SLC7A6, SLC7A7, SLC7A8, SLC7A9, SLC7A10, SLC7A11, SLC7A13, SLC7A14, SLC36A1, SLC36A2, SLC36A3, SLC36A4; SLC38A1, SLC38A2, SLC38A3, SLC38A4, SLC38A5, SLC38A6; SLC43A1, SLC43A2, or a SLC43A3 polypeptide.

10. The method of claim 9, wherein said mutant polypeptide is a SLC38A1 polypeptide.

11. A method for diagnosing arthritis as being caused by impaired amino acid transporter function in a mammal having arthritis, said method comprising:
(a) determining the level of amino acid transporter activity of a sample of cells obtained from said mammal;
(b) determining whether or not said level is less than a control level amino acid transporter activity, wherein said control level is the average amount of amino acid transporter activity of control cells from a population of healthy mammals, and wherein said healthy mammals are from the same species as said mammal; and
(c) identifying said mammal as having arthritis caused by impaired amino acid transporter function when said level is less than said control level.

12. The method of claim 11, wherein said amino acid transporter is SLC38A1.

13. A method for diagnosing arthritis as being caused by impaired amino acid transporter function in a mammal having arthritis, said method comprising determining whether or not said mammal comprises a mutant polypeptide that is part of an amino acid transporter selected from a SLC1 A1, SLC1 A2, SLC1 A3, SLC1A4, SLC1A5, SLC1A6, SLC1A7; SLC2A1, SLC2A2, SLC2A3, SLC2A4, SLC2A5, SLC2A6, SLC2A7, SLC2A8, SLC2A9, SLC2A10, SLC2A11, SLC2A12, SLC2A13, SLC2A14, SLC7A1, SLC7A2, SLC7A3, SLC7A4, SLC7A5, SLC7A6, SLC7A7, SLC7A8, SLC7A9, SLC7A10, SLC7A11, SLC7A13, SLC7A14, SLC36A1, SLC36A2, SLC36A3, SLC36A4; SLC38A1, SLC38A2, SLC38A3, SLC38A4, SLC38A5, SLC38A6; SLC43A1, SLC43A2, or a SLC43A3 polypeptide, wherein the presence of said mutant polypeptide indicates that said mammal has arthritis caused by impaired amino acid transporter function.

14. The method of claim 13, wherein said mutant polypeptide is a SLC38A1 polypeptide.

15. The method according to claim 1, wherein the arthritis is rheumatoid arthritis.

16. The method of claim 1, wherein said level of amino acid transporter activity is determined by measuring intracellular uptake of glutamine, alanine, asparagine, histidine, serine and/or cysteine.

17. The method of claim 6, wherein said level of amino acid transporter activity is determined by measuring intracellular uptake of glutamine, alanine, asparagine, histidine, serine and/or cysteine.
